SEQUENCE LISTING (2 sequences)

SEQ ID NO: 1 - protein, 15 aa, Artificial
  Synthetic peptide
GSWYAWSPLVPSAQI

SEQ ID NO: 2 - protein, 15 aa, Artificial
  Synthetic peptide
HGRFILPWWYAFSPS

(19) United States
(12) Patent Application Publication
(10) Pub. No.: US 2005/0260680 A1
(43) Pub. Date: Nov. 24, 2005

(54) Methods of diagnosing and treating autoimmune diseases

(75) Inventors: Grant Yeaman, Brentwood, TN (US); David McGuinness, Nashville, TN (US); Edward Organ, Nashville, TN (US); Kevin Osteen, Nashville, TN (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Vanderbilt University

(21) Appl. No.: 11/107,055

(22) Filed: Apr. 15, 2005

Related U.S. Application Data
(60) Provisional application No. 60/562,700, filed on Apr. 16, 2004.

(51) Int. Cl. G01N 33/53

(52) U.S. Cl. 435/7.1

(57) ABSTRACT

The present invention provides methods of diagnosing autoimmune disorders selected from the group consisting of Sjögren's Syndrome and lupus in a subject based on detecting a difference between the subject and normal individuals in the levels of individual antibody classes and/or subclasses that are specific for Tf-like antigens. The present invention also provide methods for treating autoimmune disorders selected from the group consisting of Sjögren's Syndrome and lupus in a subject comprising administering to a subject in need thereof a Tf-like antigen or an antibody thereto.

### CROSS REFERENCE

This application claims priority to U.S. Provisional Patent Application Ser. No. 60/562,700 filed Apr. 16, 2004, which is incorporated herein by reference in its entirety.

### FIELD OF THE INVENTION

The present invention relates to Tf-like antigens and antibodies thereto, diagnostic assays, therapeutic treatments, and autoimmune disorders.

### BACKGROUND OF THE INVENTION

Lupoid autoimmune diseases (“lupus”) are chronic inflammatory autoimmune diseases that can affect many organs, including skin, joints, kidneys, blood cells, heart and lungs. Discoid lupus erythematosus (DLE) is a chronic skin condition of sores with inflammation and scarring favoring the face, ears, and scalp and at times on other body areas. These lesions develop as a red, inflamed patch with a scaling and crusty appearance. When lesions occur in hairy areas such as the beard or scalp, permanent scarring and hair loss can occur. A small percentage of patients with discoid lupus can develop disease of the internal organs. Children and people with many spots are usually at more risk of this. Lupoid hepatitis (also referred to as autoimmune hepatitis) is an inflammation of the liver.

The most common form of lupus is systemic lupus erythematosus (SLE), an autoimmune disease characterized by immune dysregulation resulting in the production of antinuclear antibodies (ANA), circulating immune complexes, and complement activation. Pathologically, SLE is typically characterized by recurrent, widespread, and diverse vascular lesions. The disease has both active phases and periods of remission. Affected tissues may include the joints, skin, kidney, brain, serosa, lung, heart, and gastrointestinal tract. The disease is around 10 times more prevalent in women as compared to men and is 3 times more common in African Americans than Caucasian populations. Estimates of disease prevalence vary but are around about 0.5% of reproductive age women. Untreated SLE is not infrequently lethal.

Sjögren's syndrome (SS) is an autoimmune disease characterized by inflammation in the glands of the body. Inflammation of the lacrimal glands that produce tears leads to dry eyes and inflammation of the salivary glands leads to mouth dryness. Presence of these symptoms in the absence of manifestations typically associated with a connective tissue autoimmune disease is referred to as primary Sjögren's syndrome. Secondary Sjögren's syndrome involves not only gland inflammation, but is also associated with manifestations overlapping with other connective tissue autoimmune diseases, such as rheumatoid arthritis, SLE, or scleroderma. The disease is 10 times more common in females than males.

Systemic and organ specific autoimmune diseases lead to significant morbidity and mortality rates. Accurate and early diagnosis of these diseases allows for earlier commencement of medical management, presumably with improved clinical outcome. However, current diagnostic tests these autoimmune diseases are lacking. Currently, the best serum diagnostic test for SLE is detection by ELISA of antibodies directed against double stranded DNA. Other autoantibodies against intracellular and nuclear antigens are also measured both by ELISA and immunofluorescent cell staining, but are not specific for SLE. One of these antigens, SSA/Ro, is the most frequently positive in Sjögren's syndrome but is also often positive in SLE, and hence lacks disease specificity.

The present invention provides methods of diagnosing autoimmune disease based on a detection of autoantibody reactivity with a Tf-like antigen. The diagnostic methods of the present invention have significantly improved sensitivity and specificity compared with existing methods.

### SUMMARY OF THE INVENTION

It has been surprisingly discovered by the present inventors that Sjögren's Syndrome and lupus are associated with altered levels of autoantibodies directed to Thomsen-Friedenreich (Tf) like antigens. Accordingly, the present invention provides methods of diagnosing and treating these diseases based on the discoveries of the present invention.

In a first aspect, the present invention provides methods for diagnosing in a subject an autoimmune disease selected from the group consisting of lupus and Sjögren's syndrome, comprising detecting a difference (ie detecting an increase or decrease in antibody response) between the levels of autoantibodies in a test subject that are specific for a Tf-like antigen and the levels of such autoantibodies in the normal range. A preferred method for determining the normal range may be defined as the 99% confidence interval of the median of a group of clinically normal individuals.

In one embodiment of this aspect, the diagnosis is based on detecting a difference between the levels of individual antibody classes in a test subject that are specific for Tf-like antigens and the levels in normal individuals. In a further embodiment, the diagnosis is based on the detection of a higher anti-Tf IgG level in the test subject as compared to a normal IgG level. In another specific embodiment, the diagnosis is based on the detection of a higher level of anti-Tf IgA in the subject, as compared to the corresponding levels in a normal subject. In another embodiment, the diagnosis is based on detecting a difference between the levels of individual antibody subclasses in a test subject that are specific for Tf-like antigens and the levels in normal individuals. For example, the diagnosis can be achieved by detecting a higher level of anti-Tf IgG of a particular subclass in the test subject as compared to a normal level of this IgG subclass. Alternatively, the diagnosis can be achieved by detection of a higher level of anti-Tf IgA of a particular subclass, as compared to a normal level of this IgA subclass.

In still another embodiment of the first aspect, the diagnosis is based on detecting a difference between the levels of two or more antibody classes in a subject that are specific for Tf-like antigens, and the levels in normal individuals. Preferably, the diagnosis is achieved by detecting a higher level of anti-Tf IgG and anti-Tf IgA, as compared to the respective normal levels of these antibody classes.

In a further embodiment of this first aspect, the diagnosis is based on determining the value of a discriminant function from the levels of two or more anti-Tf antibody classes in the subject (“test value”), comparing the value to a value determined from normal levels of these antibody classes, and detecting an increase in the test value relative to the normal value as indicative of a relevant disease. Preferably, the discriminant function is derived based on the level of anti-Tf IgG and the level of anti-Tf IgA. More preferably, the discriminant function positively weighs the anti-Tf IgG level and the anti-Tf IgA level. The discriminant function can relate the IgG and IgA levels in a linear, multiplicative or geometric manner.

In another embodiment of the first aspect, the diagnosis is based on detecting a difference between the levels of two or more anti-Tf antibody subclasses in a subject and normal levels of these subclasses. For example, diagnosis can be based on a detection of a higher level of an anti-Tf IgG subclass and anti-Tf IgA subclass, as compared to the respective levels of these subclasses of antibodies in a normal population. In still another embodiment, the diagnosis is based on determining the value of a discriminant function from the levels of two or more anti-Tf antibody subclasses in the subject, comparing the test value to a normal value determined from normal levels of these antibody subclasses, and detecting an increase in the test value as indicative of the disease.

In a second aspect, the present invention further provides kits for practicing the diagnostic methods described herein. The kits can contain Tf-like antigens and means for producing a detectable signal manifesting the level of a subject's anti-Tf antibodies of a particular class or subclass. The kit can also include a sample(s) obtained from normal individual(s) or a predetermined value, and instructions for how to obtain a value from a test subject for comparison with the predetermined value. The kit can also include instructions for how to use one or more formulas of discriminant function to make the diagnosis.

In a third aspect, the present invention provides methods for treating an autoimmune disease selected from the group consisting of lupus and Sjögren's syndrome, comprising administering to a subject with one or more of lupus and Sjögren's syndrome an amount effective of a Tf-like antigen to treat the autoimmune disease. In another embodiment of this third aspect, the method comprises administering to the subject an antibody or a functional derivative or analog thereof that specifically binds Tf-like antigens. Preferably, the antibody is a humanized antibody.

### BRIEF DESCRIPTION OF THE DRAWINGS

FIG. 1 shows the mean of triplicate estimations of IgA (upper), IgG (middle) and IgM (lower) for sera from 35 normal female donors, 9 clinically confirmed SLE patients, and 10 patients with SS. Reactivities for all antibody classes were determined against synthetic serine Tf, serine 2,3 sialylated Tf, serine 2,6 sialylated Tf, and serine 2,3 and 26 disialylated Tf antigens covalently linked to polystyrene microtiter plates.

### DETAILED DESCRIPTION OF THE INVENTION

It has been surprisingly discovered by the present inventors that Sjögren's syndrome and lupus are associated with altered levels of autoantibodies directed to Thomsen-Friedenreich (Tf) like, characterized by the disaccharide structure, Gale 1-3GalNAc. For example, the present inventors have unexpectedly found that the IgG and/or IgA levels in patients having lupoid autoimmune disease are significantly higher as compared to normal individuals. The IgM levels in these patients differ variably from those of normal individuals. Based on these discoveries, the present invention provides methods of diagnosing these diseases based on detecting a difference between a test subject and normal individuals in the levels of individual antibody classes and/or subclasses that are specific for Tf-like antigens. Additionally, the present invention provides diagnostic methods based on detecting a difference between a test subject and normal individuals in the levels of one or more antibody class and/or subclass that are specific for one or more Tf-like antigens. In particular, a discriminant function analysis is provided, which reflects the differences in the levels of two or more antibody classes and/or subclasses that are specific for Tf-like antigens. Therapeutic methods based on administration of Tf-like antigens or antibodies directed against Tf-like antigens or derivatives or analogs thereof are also provided.

As used herein, a “difference” between a test subject and normal individuals in the levels of individual antibody classes and/or subclasses means detecting an increase or decrease in antibody response, between the levels of autoantibodies in a test subject that are specific for a Tf-like antigen and the levels of such autoantibodies in the normal range. A preferred method for determining the normal range may be defined as the 99% confidence interval of the median of a group of clinically normal individuals.

Altered levels of autoreactivities against Tf-like antigens have been observed with patients having endometriosis, as described in U.S. Pat. No. 6,645,725. However, the present invention provides for the first time that altered levels of autoreactivities against Tf-like antigens are also associated with Sjögren's Syndrome and lupus.

Autoimmune diseases arise when a patient's immune system fails to discriminate between self and non-self and starts to react against self antigens. The autoreactivity may be in the form of T-cells or antibodies. Currently, diagnosis of the different autoimmune diseases relies on the interpretation of a series of overlapping clinical symptoms, such as Raynaud's phenomenon, sclerodactyly and alveolitis and the presence of an overlapping pattern of different autoantibodies which are typically directed against nucleic acids or proteins (often ones found in the nucleus of cells). Thus, these are features common to a number of autoimmune diseases, even though these features cannot be used on their own to define a particular disease. For example, although double stranded DNA specific antibodies are regarded as diagnostic for SLE, patients with rheumatoid arthritis, Sjögren's syndrome, mixed connective tissue disease, progressive systemic sclerosis and discoid lupus erythematosus may also be positive. It is now generally thought that antibodies to Ro(SS-A) and La(SS-B), occur in patients with Sjögren's syndrome who also have features of SLE.

Without intending to be bound by any particular theory, it is further proposed that the elevated IgG and IgA responses observed in patients with Sjögren's Syndrome and lupus are due to a switch in the patients' antibody responses from T independent to T dependent responses. Anti-Tf antibodies are present in normal individuals. Similar to antibodies specific for other carbohydrate antigens such as ABO blood group antigens, anti-Tf antibodies are regarded as “natural” antibodies. Natural antibodies are typically produced by B 1 a cells in a T cell independent manner, and are typically low affinity pentameric IgM and dimeric IgA antibodies. It is hypothesized that in Sjögren's Syndrome and lupus, the Tf-like antigens are being presented as a glycopeptide in the context of MHC class II. The switch from T independent antibody responses to T dependent antibody responses therefore results in elevated IgG and IgA levels. IgM levels could remain essentially the same since IgM production could occur by either mechanism.

As used herein, the term “Tf antigen” refers to an antigen or antigenic epitope characterized by the Galβ1-3GalNAc disaccharide moiety. The term “Tf-like antigen” encompasses molecules or compounds that comprise or consist of the Tf antigen or analogs or functional derivatives of the Tf antigen, as well as molecules or compounds that contain (i.e., include) one or more units of the Tf antigen or analogs or functional derivatives thereof.

By “analog or functional derivative of the Tf antigen” is meant a compound that is analogous to, or derived from, the disaccharide moiety of Galβ1-3GalNAc, and that binds to one or more antibodies or molecules that are known to specifically bind the Tf antigen. Examples of antibodies or molecules known to specifically bind the Tf-antigen include jacalin (a lectin present in the seeds of the Jackfruit, Artocarpus integrifolia), monoclonal antibodies 49H.8, (Rahman and Longenecker, 1982, J. Immun. 129(5): 2021-4), 155H7 and 170H82 (Longenecker et al. 1987, J. Nat. Cancer Inst., 78(3): 489-96), A78-G/A7 (Karsten et al. 1995, Hybridoma 14(1): 37-44), HB-T1 (DAKO Co.), RS1-114 and AHB-25B (Stein et al. 1989, Cancer Res. 49(1): 32-7), HT8 (Metcalfe et al., 1984, Br. J. Cancer 49(3): 337-42), 161H4 (Longenecker et al., 1987), HH8 (Clausen et al. 1988), and BW835 (Hanish et al, 1995, Cancer Res. 55(18): 4036-40). Examples of derivatives of the Tf antigen include a sialylated Tf antigen (i.e. Tf antigen with a sialic acid residue attached to the galactosyl and/or galactosamine residues) and sulfations of the galactosyl residue.

As used herein, Tf-like antigens such as a compound or molecule “comprising” or “containing” the Tf antigen or analogs thereof, can be a naturally-occurring molecule, e.g., an autoantigen present in humans or other mammals such as human α₂-Heremans Schmidt glycoprotein (α₂-HSG) and bovine fetuin (the bovine homolog of human α₂-HSG). Tf-like antigens can also be synthetic molecules, for example, a naturally occurring molecule which does not contain the Tf-antigen but is synthetically linked to the Tf-antigen or analog thereof, or a molecule containing the Tf antigen which molecule is wholly designed and made synthetically.

The term “diagnosing a disease” means to determine the presence or the likelihood of the presence of the disease in a subject. A combination of a detection based on autoreactivities against Tf-like antigens with conventional diagnosis (i.e., based on clinical syndromes and autoantibodies against other autoantigens) achieves a more definitive diagnosis of a particular disease in the patient. For example, SLE is typically characterized by recurrent, widespread, and diverse vascular lesions. Affected tissues may include the joints, skin, kidney, brain, serosa, lung, heart, and gastrointestinal tract. SS is typically characterized by inflammation in the glands of the body. Inflammation of the lacrimal glands that produce tears leads to dry eyes and inflammation of the salivary glands leads to mouth dryness.

For the purpose of diagnosis in accordance with the methods provided herein, the levels of autoantibodies in a subject can be determined by taking any antibody-containing tissue or body fluid from the subject. Antibody-containing tissue includes tissue manifesting abnormalities. Antibody-containing fluid includes but is not limited to, e.g., blood, serum, plasma, saliva, tears, sweat, peritoneal fluid, and vaginal secretions. In a preferred embodiment, antibody-containing body fluid from a test subject, in particular, serum, is employed.

The terms “anti-Tf antibody”, “antibody directed against Tf-like antigens”, and “antibody specific for Tf-like antigens” are used herein interchangeably to refer to an antibody specific for epitopes characterized by the Galβ1-3GalNAc disaccharide moiety. The specificity of such an antibody is evidenced, e.g., by its binding to one or more molecules or compounds which contain the Galβ1-3GalNAc disaccharide moiety, and its lack of binding to otherwise the same molecule or molecules but devoid of the disaccharide moiety.

In one embodiment, the present invention provides a method of diagnosing an autoimmune disorder selected from the group consisting of Sjögren's syndrome and lupus in a subject by detecting a difference between the levels of individual antibody classes and/or subclasses in the subject that are specific for Tf-like antigens and normal levels of the respective antibody classes and/or subclasses.

The diagnosis can be achieved by detecting a difference between the levels of individual antibody classes specific for Tf-like antigens in a subject and normal levels. For example, the detection of a higher level of anti-Tf IgG in the test subject as compared to a normal IgG level is indicative of a relevant disease (i.e., Sjögren's syndrome and/or lupus) in the subject. Alternatively, the detection of an increase in the level of anti-Tf IgA, as compared to a normal IgA level is also indicative of a relevant disease in the subject.

The diagnosis can also be achieved by detecting a difference between the levels of individual antibody subclasses specific for Tf-like antigens in a subject and normal levels of these subclasses. For example, the detection of a higher level of anti-Tf IgG of a particular subclass in the test subject as compared to a normal level of this IgG subclass is indicative of a relevant disease in the subject. Alternatively, the detection of a higher level of anti-Tf IgA of a particular subclass, as compared to a normal level of this IgA subclass, is also indicative of a relevant disease in the subject.

A normal antibody level can be a single value, such as a median or mean level determined from a group or population of normal individuals, i.e., individuals without the disease. A normal antibody level can also be a range of values determined from normal individuals. A normal antibody level can be determined using tissue or body fluid samples from normal individuals in assays run side-by-side with the assay on a test sample. Alternatively, a normal antibody level is predetermined or established. In such instance, the antibody level of a test subject should be determined in the same manner (i.e., the same tissue or fluid source and the same assay) as the normal antibody level. A preferred method for determining the normal range may be defined as the 99% confidence interval of the mean of a group of clinically normal individuals, where the 99% confidence interval is calculated as the mean of the population plus or minus three standard deviations.

In a preferred embodiment, the invention provides methods of diagnosing in a subject an autoimmune disease selected from the group consisting of lupus and Sjögren's Syndrome, comprising:
a) contacting an antibody-containing tissue sample or an antibody-containing fluid sample from a subject suspected of or at risk of having an autoimmune disease selected from the group consisting of lupus and Sjögren's syndrome, with an antigen comprising a Tf-like antigen;
b) detecting autoantibody reactivity of the sample to the antigen; and
c) determining a level of autoantibody reactivity of the sample relative to a normal antibody level, and
d) correlating the level of autoantibody reactivity of the sample with a diagnosis of a presence or absence in the subject of an autoimmune disease selected from the group consisting of lupus and Sjögren's syndrome.

As used herein, a subject that is suspected of or at risk of having an autoimmune disease include those subjects that exhibit symptoms characteristic of lupus and Sjögren's syndrome; those that have previously exhibited symptoms characteristic of lupus and Sjögren's syndrome; those that were previously been diagnosed with lupus and Sjögren's syndrome but have since become asymptomatic; those that are genetically predisposed to lupus and Sjögren's syndrome.

Symptoms of lupus include, but are not limited to, inflammation of the skin, joints, kidneys, blood cells, heart and/or lungs.

Symptoms of discoid lupus erythematosus (DLE) include but are not limited to chronic skin condition of sores with inflammation and scarring of the face, ears, and scalp; inflamed lesions of these areas with a scaling and crusty appearance; internal organ inflammation. Lupoid hepatitis (also referred to as autoimmune hepatitis) is characterized by inflammation of the liver. Systemic lupus erythematosus (SLE) is characterized by immune dysregulation resulting in the production of antinuclear antibodies (ANA), circulating immune complexes, and complement activation, and pathological symptoms recurrent, widespread, and diverse vascular lesions; affected tissues may include the joints, skin, kidney, brain, serosa, lung, heart, and gastrointestinal tract. SLE has both active phases and periods of remission, and is approximately 10 times more prevalent in women as compared to men, and 3 times more common in African Americans than Caucasian populations.

Symptoms of Sjögren's syndrome (SS) include inflammation in the glands of the body. Inflammation of the lacrimal glands that produce tears leads to dry eyes and inflammation of the salivary glands leads to mouth dryness. Presence of these symptoms in the absence of manifestations typically associated with a connective tissue autoimmune disease is referred to as primary Sjögren's syndrome. Secondary Sjögren's syndrome involves not only gland inflammation, but is also associated with manifestations overlapping with other connective tissue autoimmune diseases, such as rheumatoid arthritis, SLE, or scleroderma. The disease is 10 times more common in females than males.

Thus, in one embodiment, the diagnosing comprises diagnosing the presence of one or more autoimmune diseases selected from the group consisting of lupus and Sjögren's Syndrome when the level of autoantibody reactivity of the sample is greater than a normal antibody level. In an alternative embodiment, the diagnosing comprises diagnosing an absence of an autoimmune disease selected from the group consisting of lupus and Sjögren's Syndrome when the level of autoantibody reactivity of the sample is similar to control (ie: falls within the 99% confidence interval for the normal population). In further preferred embodiments, the detecting comprises detecting of one or both of anti-Tf-IgG and anti-Tf IgA autoantibody reactivity of the sample to the antigen; the determining comprises determining a level of one or both of anti-Tf-IgG and anti-Tf IgA autoantibody reactivity of the sample relative to a normal antibody level; and the correlating comprises correlating the level of one or both of anti-Tf-IgG and anti-Tf IgA autoantibody reactivity of the sample with a diagnosis of a presence or absence in the subject of an autoimmune disease. In these embodiments, it is further preferred that the diagnosing comprises diagnosing a presence of an autoimmune disease selected from the group consisting of lupus and Sjögren's syndrome when the level of one or both of anti-Tf-IgG and anti-Tf IgA autoantibody reactivity of the sample is greater than control. Alternatively, the diagnosing comprises diagnosing an absence of an autoimmune disease selected from the group consisting of lupus and Sjögren's syndrome when the level of one or both of anti-Tf-IgG and anti-Tf IgA autoantibody reactivity of the sample is similar to control.

In a preferred embodiment, the autoimmune disease is lupus, and is selected from systemic lupus erythematosus (SLE), discoid lupus, and lupoid hepatitis. In a more preferred embodiment, the lupus comprises SLE.

In a further preferred embodiment, the autoimmune disease is Sjögren's syndrome, including primary or secondary Sjögren's syndrome.

In further preferred embodiments of all of the above embodiments, Tf-like antigen comprises one or more Galβ1-3GalNAc disaccharide moiety.

In another embodiment, the present invention provides a method of diagnosing an autoimmune disorder selected from the group consisting of Sjögren's syndrome and lupus in a subject by detecting a difference between the levels of two or more antibody classes in the subject that are specific for Tf-like antigens, and normal levels of these antibody classes.

Preferably, the levels of IgG and IgA are detected and compared to a normal IgG level and a normal IgA level. Diagnosis is achieved by detecting a higher level of anti-Tf IgG and/or a higher level of anti-Tf IgA, as compared to the respective normal levels of these antibody classes.

In a further embodiment (and in a preferred embodiment of the above embodiments), the present invention provides a method of diagnosing an autoimmune disorder selected from the group consisting of Sjögren's syndrome and/or lupus in a subject by determining the value of a discriminant function based on the levels of two or more anti-Tf antibody classes in the subject, comparing the value to a value determined based on normal levels of these antibody classes, and detecting an increase in the value as indicative of lupus and/or Sjögren's syndrome.

As used herein, the term “discriminant function” refers to a function that weighs levels of multiple, i.e., two or more, classes or subclasses of anti-Tf antibodies so as to reflect the net changes in the classes or subclasses, thereby improving the differentiation of disease individuals from normal individuals.

For example, a discriminant function can be based on the level of anti-Tf IgG and the level of anti-Tf IgA.

In a preferred embodiment of the present invention, a discriminant function positively weighs anti-Tf IgG levels and anti-Tf IgA level, such that a comparison of the value of the discriminant function from a patient having Sjögren's Syndrome and/or lupus to a value determined from normal individuals would reflect the differences between the subject and normal individuals in both IgG and IgA levels. Such a discriminant function permits diagnosis to be carried out at higher specificity and sensitivity, and is therefore desirable especially when the differences in individual antibody classes are not substantial.

By “sensitivity” is meant the fraction of patients with Sjögren's Syndrome and/or lupus correctly identified by a test.

By “specificity” is meant the fraction of individuals without Sjögren's Syndrome and/or lupus correctly identified by a test.

A discriminant function can weight individual values of IgM, IgG and IgA levels for a particular antigen to maximize the separation of the normals, SLE and SS. An example of such a discriminant function that relates IgM, IgG and IgA levels is given in the following formula:
Discriminant value=−0.138(2,3 sialyl TF IgA)−1.470(2,6 sialyl TF IgA)−2.136(2,6 sialyl TF IgM)+1.466(2,3,2,6 disialyl TF IgG)+1.9346(2,3,2,6 disialyl TF IgM)+2.605(TF IgA)−1.569(TF IgG)

Similarly, a second discriminant function is used to differentiate SLE from SS using a similar equation to the one above but using the coefficients shown in the right hand column of Table 1:

TABLE 1
Discriminant Function Coefficients for differentiating
between Normals, SLE and SS
Standardized Coefficients

| | Normal vs | |
|---|---|---|
| | SLE & SS | SLE vs SS |
| 2,3 sialyl TF IgA | −0.138 | 3.147 |
| 2,6 sialyl TF IgA | −1.470 | −1.144 |
| 2,6 sialyl TF IgM | −2.136 | −2.551 |
| 2,3,2,6 disialyl TF IgG | 1.466 | 1.390 |
| 2,3,2,6 disialyl TF IgM | 1.9346 | 2.185 |
| TF_IgA | 2.605 | −1.950 |
| TF_IgG | −1.569 | −1.052 |

In the first equation a positive value is an indicator for disease.

As used herein, 2,3 sialyl Tf means antibodies that selectively bind to the Tf-like antigen 2,3 sialyl.

As used herein, “2,6 sialylTf IgA” means antibodies of the IgA class that selectively bind to the Tf-like antigen 2,6 sialylTf.

As used herein, “2,6 sialyl Tf IgM” means antibodies of the IgM class that selectively bind to the Tf-like antigen 2,6 sialyl Tf As used herein, “2,3,2,6 disialyl Tf IgG” means antibodies of the IgG class that selectively bind to the Tf-like antigen 2,3,2,6 disialyl Tf.

As used herein, “2,3,2,6 disialyl Tf IgM” means antibodies of the IgM class that selectively bind to the Tf-like antigen 2,3,2,6 disialyl Tf.

As used herein, Tf IgA means antibodies of the IgA class that bind the Tf antigen.

As used herein, Tf IgG means antibodies of the IgG class that bind the Tf antigen.

As noted in the Examples below, this discriminant function differentiates between normal individuals and SLE. After applying this formula to the values obtained on antibody reactivity, a positive value indicates SLE, whereas a zero or negative value indicates no disease. There are similar discriminant functions that can be used to differentiate between SLE and SS, and SS and normal individuals. For example, the elevated IgA responses shown in the Examples that follow for SS show complete separation from those seen in the SLE group. Thus, these two diseases can be differentially diagnosed in the same set of immunometric estimations. Distinction between SLE, SS, and normal groupings can be made by setting cut off values for IgG and/or IgA and/or IgM, based on range against 1 or more controls.

Alternatively, a discriminant function can relate IgM, IgG, IgA, levels in a multiplicative or geometric manner.

According to the present invention, the value of a discriminant function can be determined based on the IgM, IgG and IgA levels of a test subject. Such value is then compared to a value determined based on normal IgM, IgG and IgA levels, i.e., IgG and IgA levels of normal individuals. When comparing the value of a discriminant function derived form a test subject to a value determined from normal individuals, an increase in the value is indicative of a relevant disease in the test subject.

It should be noted that the value determined from normal individuals can be a single value, such as a median or mean value, or a range of values. Such value can be determined by employing tissue or body fluid samples from normal individuals in assays run side-by-side with the assay on a test sample. Alternatively, such value can be a predetermined value or range of values, in which case, the discriminant function value of a test subject should be determined in the same manner as the value determined from normal individuals—that is, using the same assay for determining the IgG and IgA levels, and the same formula of discriminant function.

In another embodiment, the present invention provides a method of diagnosing an autoimmune condition selected from Sjögren's syndrome and/or lupus in a subject by detecting a difference between the levels of two or more anti-Tf subclasses in a subject and normal levels of these subclasses, i.e., the levels of these subclasses of antibodies in a normal population.

Preferably, the two subclasses of antibodies include an IgG subclass and an IgA subclass, where the diagnosis is based on the detection of a higher level of the IgG subclass and a lower level of the IgA subclass in the subject as compared to normal levels of these subclasses.

In connection with this embodiment, a discriminant function can be derived based on the levels of certain subclasses, for example, certain IgG subclass or subclasses and certain IgA subclass or subclasses. For example, a discriminant function can positively weigh the level(s) of certain IgG and IgA subclass or subclasses. Similar to a discriminant function that weighs levels of IgG and IgA classes, a discriminant function that weighs levels of subclasses can relate levels of subclasses in a linear, multiplicative or geometric manner.

The value of a discriminant function based on the levels of subclasses of anti-Tf autoantibodies in a test subject is then compared to a value determined based on levels of the subclasses in normal individuals. An increase in the value of the discriminant function is correlated with a diagnosis of a relevant disease in the subject.

Also contemplated in the present invention are methods of diagnosis based on detecting the levels of a combination of one or more antibody classes with one or more antibody subclasses of a different antibody class.

The levels of IgG, IgA or subclasses of IgG or IgA that are specific for Tf-like antigens, can be determined by using any of the assays, typically immunoassays, known in the art. A typical immunoassay employs an antibody-containing tissue or body fluid sample from a subject, Tf-like antigens, and means for producing a detectable signal manifesting the level of a particular antibody class or subclass.

Tf antigens or molecules containing the Tf-antigen for use in an assay may be obtained from various sources. For example, molecules containing the Tf-antigen may be purified from conditioned culture medium used to cultivate tumor cell lines such as the adenocarcinoma cell line LS174T, obtainable through the American Type Culture Collection (ATCC). Transitional cell carcinoma lines may also serve as source of molecules containing the Tf antigen. The molecules containing the Tf antigen can be purified from such conditioned culture medium by affinity chromatography using CnBr activated Sepharose column conjugated with an antibody specific to Tf-like antigens, e.g., MAb 49H.8. Gel filtration can also be performed for additional purification. Still further purification of molecules containing the Tf antigen can be achieved by utilizing lectin affinity chromatography with insolubilized Jackfruit lectin (Jacalin) or other lectin. Jacalin may be obtained from Vector laboratories, Burlingame, Calif.

Purified naturally occurring Tf-like antigens, such as IgA, hemopexin, alpha-2-Heremans Schmidt, and bovine fetuin, all of which bear the Tf-antigen naturally, can also be used in the assay. A preferred source of Tf-like antigens is commercially synthesized Tf antigen or analog or functional derivative, covalently linked to bovine serum albumin or polyacrylamide or Serine or Threonine The Tf antigen and its glycoforms are available from commercial vendors such as BioCarb as recently described by Dahlenborg et al. (1997) In. J. Cancer 70:63-71. Alternatively, Tf-like antigens may be obtained by custom synthesis from commercial vendors such as Carbohydrate Synthesis (Oxford, UK).

For determining the levels of anti-Tf IgG, IgA or subclasses thereof, an immunoassay can be carried out in a variety of formats such as, e.g., enzyme linked immunoabsorbent assay (ELISA), fluorescent immunosorbent assay (FIA), chemical linked immunosorbent assay (CLIA), radioimmuno assay (RIA), and immunoblotting. For a review of the different immunoassays which can be used in the present invention, see, The Immunoassay Handbook, David Wild, ed., Stockton Press, New York, 1994; and U.S. Pat. No. 6,645,725, the disclosure of which is incorporated herein by reference. Even though the particular format of the immunoassay is not critical to the present invention, immunometric assays for antibody testing are preferred. See, The Immunoassay Handbook, supra, chapter 2.

In an immunometric assay, Tf-like antigen is immobilized on a solid support or surface such as a bead, plate, slide or microtiter dish. An aliquot of an antibody-containing sample from a subject is added to the solid support and allowed to incubate with the Tf-like antigen in a liquid phase. An antibody that recognizes a constant region in human autoantibodies present in the sample is added. This antibody is an anti-human antibody and is also part of a signal producing system. For example, the anti-human antibody can be specific for the heavy chain constant regions of a particular antibody class (e.g., IgA, IgG, or IgM) or subclass. After separating the solid support from the liquid phase, the support phase is examined for a detectable signal, which represents the amount of anti-Tf autoantibodies of the particular class or subclass present in the sample.

The signal producing system is made up of one or more components, at least one of which is a label, which generate a detectable signal that relates to the amount of antibody molecules bound and/or unbound to the Tf-like antigen. The label is a molecule that produces or which may be induced to produce a signal. Examples of labels include fluorescent, enzymatic, chemiluminescent, photosensitive or suspendable particles. The signal is detected and may be measured by detecting enzyme activity, luminescence or light absorbance. Radiolabels may also be used and levels of radioactivity detected and measured using a scintillation counter.

Examples of enzymes which can be used to label the anti-human immunoglobulin include β-D-galactosidase, horseradish peroxidase, alkaline phosphatase, and glucose-6-phosphate dehydrogenase (“G6PDH”). Examples of fluorescent molecules which may be used to label the anti-human immunoglobulin include fluorescein, isothiocyanate, rhodamine compounds, phycoerythrin, phycocyanin, allophycocyanin, o-phthaldehyde and fluorescamine. Chemiluminescers include e.g., isoluminol.

Free (i.e., unbound) labeled antibody is separated from bound antibody and if necessary, an appropriate substrate with which the label, e.g., enzyme, reacts is added and allowed to incubate.

In a preferred assay, the anti-human antibody is labeled with an enzyme, e.g., either horseradish peroxidase or alkaline phosphatase.

The amount of color, fluorescence, luminescence, or radioactivity present in the reaction (depending on the signal producing system used) is proportionate to the amount of autoantibodies in a patient's sample that are specific for the Tf-like antigen. Quantification of optical density may be performed using spectrophotometric methods. Quantification of radiolabel signal may be performed using scintillation counting and/or gamma counting.

Enzymes may be covalently linked to Tf-like antigen reactive antibodies for use in the methods of the invention using well known techniques. For example, alkaline phosphatase and horseradish peroxidase may be conjugated to antibodies using glutaraldehyde. Horseradish peroxidase may also be conjugated using the periodate method. Commercial kits for enzyme conjugating antibodies are widely available. Enzyme conjugated anti-human and anti-mouse immunoglobulin specific antibodies are available from multiple commercial sources. Biotin labeled antibodies may be used as an alternative to enzyme linked antibodies. In such cases, bound antibody would be detected using commercially available streptavidin-horseradish peroxidase or streptavidin-alkaline phosphatase detection systems.

Enzyme labeled antibodies produce different signal sources, depending on the substrate. Signal generation involves the addition of substrate to the reaction mixture. Common peroxidase substrates include ABTS®(2,2′-azinobis(ethylbenzothiazoline-6-sulfonate)), OPD (O-phenylenediamine) and TMB (3,3′, 5,5′-tetramethylbenzidine). These substrates require the presence of hydrogen peroxide. p-nitrophenyl phospate is a commonly used alkaline phosphatase substrate. During an incubation period, the enzyme gradually converts a proportion of the substrate to its end product. At the end of the incubation period, a stopping reagent may be added which stops enzyme activity. Signal strength is determined by measuring optical density, usually via spectrophotometer.

Alkaline phosphatase labeled antibodies may also be measured by fluorometry. Thus in the immunoassays of the present invention, the substrate 4-methylumbelliferyl phosphate (4-UMP) may be used. Alkaline phosphatase dephosphorylates 4-UMP to form 4-methylumbelliferone (4-MU), the fluorophore. Incident light is at 365 nm and emitted light is at 448 nm.

In a second aspect of the present invention, kits are provided for diagnosing Sjögren's syndrome and/or lupus in a subject in accordance with the methods described herein.

In one embodiment, the kit contains Tf-like antigens and means for producing a detectable signal manifesting the levels of a subject's anti-Tf antibodies of two or more classes or subclasses. Such means include, e.g., antibodies known to bind Tf-like antigens, with or without a label; antibodies that bind to a known antibody, typically with a label; antibodies, typically with a label, that recognize certain class or subclass of human immunoglobulins; reagents that facilitate the detection of a signal, among others.

The kit can also include a control sample, e.g., a collection of tissue or body fluid samples from normal individuals such as a pooled serum sample from multiple healthy individuals. Alternatively, the kit can include a predetermined value derived from antibody levels in normal individuals, and instructions for how to obtain a value from a test subject for comparison with the predetermined value.

The kit can also include instructions for use of one or more formulas of discriminant function make the diagnosis.

In a third aspect of the present invention, therapeutic methods for treating Sjögren's syndrome and/or lupus in a patient are provided.

By “treating” is meant preventing, inhibiting or ameliorating the symptoms associated with a relevant disease.

Methods of treating endometriosis based on administration of Tf-like antigens or an antibody to Tf-like antigens have been described in U.S. Patent Publication 20040001823. However, prior to the present invention, there has been no suggestion to treat Sjögren's syndrome and/or lupus by administering Tf-like antigens or an antibody to a patient in need thereof.

Without intending to be bound to any particular theory, it is proposed in accordance with the present invention that endometriosis, Sjögren's syndrome and/or lupus are diseases with a common etiology but different organ specificities. This hypothesis is based on several observations. In the first instance, it has been observed that sex steroid hormones play a major role in endometriosis, SLE and SS, as evidenced by the tenfold greater prevalence in females than males for SLE and Sjögren's and the well documented estradiol dependence of endometriosis. Furthermore, it has been observed that increased stromal fibroblast activation is common to all three diseases. Moreover, it has been observed that incidence of SLE and Sjögren's are 20 times more prevalent in women with endometriosis compared to women without endometriosis (Sinii et al., Human Reproduction 17 (10): 2715-2724, 2002). Additionally, anti-TF antibodies of the IgG class are elevated in both diseases. Accordingly, as provided in the present invention, Sjögren's Syndrome and/or lupus can also be treated based on administration of Tf-like antigens or an antibody specific for Tf-like antigens.

In one embodiment, the present invention provides a method of treating an autoimmune disorder selected from the group consisting of SS and lupus by administration into a patient of a purified antibody or a functional derivative or analog thereof which specifically binds Tf-like antigens. In a preferred embodiment, the method of treating an autoimmune disease selected from the group consisting of lupus and Sjögren's syndrome, comprises administering to a subject with one or more of lupus and Sjögren's syndrome an amount effective to treat the autoimmune disease of an antibody that specifically binds a Tf-like antigen.

By “a functional derivative or analog” of an antibody is meant a derivative or molecule that specifically binds Tf-like antigens and possesses the antigen binding domain of a Tf-specific antibody or a structure that mimics the antigen binding domain of a Tf-specific antibody. Examples of functional antibody derivatives or analogs include Fab fragments, Fv fragments (composed of the VH and VL domains of an antibody), VH domains (Ward et al., Nature 341, 554-546, 1989), single chain Fv antibodies (“scFv”, composed of the variable regions of the immunoglobulin heavy and light chain covalently connected by a peptide linker; Hu et al., Cancer Research 56, 3055-3061, 1996). Functional antibody derivatives or analogs can also include engineered protein scaffold, for example, “minibodies” such as the one designed by Pessi et al. (Nature 362, 3678-369, 1993) using a part of the Ig VH domain as the template; “diabodies”, which are small bivalent and bispecific antibody fragments and are composed of a heavy-chain variable domain (VH) connected to a light-chain variable domain (VL) in the same polypeptide (P. Holliger et al., Proc. Natl. Acad. Sci., U.S.A. 90, 6444-6448, 1993). Additional examples of antibody analogs include molecules engineered based on a scaffold protein such as a fibronectin type III (Fn3) polypeptide (Koide et al., Dept. of Biochemistry and Biophysics, University of Rochester Medical Center, Rochester, N.Y. 14642. FASEB J. 11 No. 9, PA1155, 1997; Koide et al., J. Mol. Biol., 284, 1141-1151, 1998; U.S. Pat. No. 6,462,189 B1) and members of the lipocalin (Weiss et al., Chemistry & Biology, 7:R177-R184). Furthermore, antibody analogs can include molecules selected from large combinatorial libraries by techniques such as phage display and mRNA display (Wilson et al., Proc. Natl. Acad. Sci., U.S.A. 98(7):3750-5 2001, WO 01/64942 A1, and WO 02/32925 A2). For example, Tf-antigen specific peptides, P-10 (GSWYAWSPLVPSAQI) (SEQ ID NO:1) and P-30 (HGRFILPWWYAFSPS) (SEQ ID NO:2), have been isolated from a bacteriophage display library (Peletskaya et al., J. Mol. Biol. 270, 374-384, 1997; Glinsky et al., Cancer Research 61, 4851-4857, 2001).

Suitable antibodies for use in the therapeutic methods of the present invention include both polyclonal and monoclonal antibodies known to bind to Tf-like antigens specifically, e.g., monoclonal antibodies 49H.8, (Rahman and Longenecker, 1982, J. Immun. 129(5): 2021-4), 155H7 and 170H82 (Longenecker et al. 1987, J. Nat. Cancer Inst., 78(3): 489-96), A78-G/A7 (Karsten et al. 1995, Hybridoma 14(1): 37-44), HB-T1 (DAKO Co.), RS1-114 and AHB-25B (Stein et al. 1989, Cancer Res. 49(1): 32-7), HT8 (Metcalfe et al., 1984, Br. J. Cancer 49(3): 337-42), 161H4 (Longenecker et al., 1987), HH8 (Clausen et al. 1988), and BW835 (Hanish et al, 1995, Cancer Res. 55(18): 4036-40).

In a preferred embodiment of the present invention, the antibody for use in the present methods is a humanized antibody. By “a humanized antibody” is meant an antibody that is encoded by a nucleotide sequence which includes at least a portion of a human immunoglobulin gene sequence. Humanized antibodies include mouse-human chimeric antibodies which contain the variable region of a murine mAb, joined to the constant regions of a human immunoglobulin. Chimeric antibodies and methods for their production are known in the art. See, e.g., Cabilly et al., European Patent Application 125023; Taniguchi et al., European patent Application 171496; Morrison et al., European Patent Application 173494; Neuberger et al., PCT Application WO 86/01533; Kudo et al., European Patent Application 184187; Robinson et al., International Patent Publication WO8702671; Liu et al., Proc. Natl. Acad. Sci. USA 84:3439-3443 (1987); Sun et al., Proc. Natl. Acad. Sci. USA 84:214-218 (1987); Better et al., Science 240:1041-1043 (1988). These references are incorporated herein by reference. Generally, DNA segments encoding the heavy and light chain antigen-binding regions of the murine mAb can be cloned from the mAb-producing hybridoma cells, which can then be joined to DNA segments encoding C_{H} and C_{L} regions of a human immunoglobulin, respectively, to produce murine-human chimeric immunoglobulin-encoding genes. Humanized antibodies can also be made using the conformational construction approach described in, e.g, Maeda et al., Hum. Antibod. Hybridomas 2: 124-134, 1991, and Padlan, Mol. Immunol. 28: 489-498, 1991. Alternatively, humanized polyclonal antibodies can be made from transgenic animals containing human immunoglobulin gene sequences, as disclosed in e.g., International Application No. WO2000046251.

In accordance with the present invention, the antibody or a functional derivative or analog can be administered to a patient in combination with a pharmaceutically acceptable carrier. The carrier can be liquid, semi-solid, e.g. pastes, or solid carriers. Except insofar as any conventional media, agent, diluent or carrier is detrimental to the recipient or to the therapeutic effectiveness of the antibody contained therein, its use in practicing the methods of the present invention is appropriate. Examples of carriers include fats, oils, water, saline solutions, lipids, liposomes, resins, binders, fillers and the like, or combinations thereof. The carrier for use in the present methods also include a controlled release matrix, a material which allows a slow release of substances mixed or admixed therein. Examples of such controlled release matrix material include, but are not limited to, sustained release biodegradable formulations described in U.S. Pat. No. 4,849,141, U.S. Pat. No. 4,774,091, U.S. Pat. No. 4,703,108, and Brem et al. (J. Neurosurg. 74: 441-446, 1991), all of which are incorporated herein by reference.

In accordance with the present invention, Tf-like antigens specific antibody or a functional derivative or analog can be combined with the carrier in any convenient and practical manner, e.g., by solution, suspension, emulsification, admixture, encapsulation, absorption and the like, and if necessary, by shaping the combined compositions into pellets or tablets. Such procedures are routine for those skilled in the art.

The amount of an antibody or a functional derivative or analog to be therapeutically effective depends on the activity of the antibody or the functional derivative or analog and other clinical factors, such as weight and condition of the subject, the subject's response to the therapy, the type of formulations and the route of administration. The precise dosage of an antibody or a functional derivative or analog to be therapeutically effective can be determined by those skilled in the art. As a general rule, the therapeutically effective dosage of an antibody or a functional derivative or analog can be in the range of about 0.5 μg to about 2 grams per unit dosage form. A unit dosage form refers to physically discrete units suited as unitary dosages for treatment: each unit containing a pre determined quantity of the active material calculated to produce the desired therapeutic effect in association with any required pharmaceutical carrier. The methods of the present invention contemplate single as well as multiple administrations, given either simultaneously or over an extended period of time.

Antibodies specific for Tf-like antigens or functional derivatives or analogs can be administered via standard routes, including the oral, ophthalmic nasal, topical, parenteral injections (e.g., intravenous, intraperitoneal, intradermal, subcutaneous or intramuscular), as well as direct injection to a pre-selected tissue site.

In another embodiment, the present invention provides a method of treating an autoimmune disease selected from the group consisting of lupus and Sjögren's syndrome, comprises administering to a subject with one or more of lupus and Sjögren's syndrome an amount effective to treat the autoimmune disease of one or more Tf-like antigens.

According to the present invention, Tf-like antigens suitable for use in the therapeutic methods of the present invention include those described hereinabove in connection with the diagnostic methods of the present invention. For example, Tf-like antigens may be purified from conditioned culture medium used to cultivate tumor cell lines such as the adenocarcinoma cell line LS 174T, obtainable through the American Type Culture Collection (ATCC), or transitional cell carcinoma lines. Tf-like antigen may be purified from such conditioned culture medium by affinity chromatography using an MAb 49H.8-CnBr activated Sepharose column. Gel filtration may also be performed for additional purification. Still further purification of Tf-like antigen may be achieved by utilizing lectin affinity chromatography with insolubilized peanut agglutinin (PNA) or other lectin.

Purified serum proteins bearing the Tf antigen or analogs or functional derivatives, such as IgA, hemopexin, and alpha-2-Heremans Schmidt, may also be used as Tf-like antigens. A preferred source of Tf-like antigens is commercially synthesized Tf antigen or analogs or functional derivatives, covalently linked to human serum albumin. The Tf antigen and its glycoforms are available from commercial vendors such as BioCarb as recently described by Dahlenborg et al. (1997) In. J. Cancer 70:63-71. Alternatively, Tf-like antigens may be obtained by custom synthesis from commercial vendors such as Carbohydrate Synthesis (Oxford, UK).

In a preferred embodiment, Tf-like antigens that contain multiple units of the disaccharide structure, Galβ1-3GalNAc or analogs or functional derivatives, is employed in the administration to a patient.

According to the present invention, Tf-like antigens can be administered to a patient in combination with a pharmaceutically acceptable carrier. Suitable pharmaceutically acceptable carriers have been described hereinabove.

The amount of Tf-like antigens to be therapeutically effective depends on the certain clinical factors, such as weight and condition of the subject, the subject's response to the therapy, the type of formulations and the route of administration. The precise dosage of Tf-like antigens to be therapeutically effective can be determined by those skilled in the art. As a general rule, the therapeutically effective dosage of Tf-like antigens can be in the range of about 0.5 μg to about 2 grams per unit dosage form. A unit dosage form refers to physically discrete units suited as unitary dosages for treatment: each unit containing a pre determined quantity of the active material calculated to produce the desired therapeutic effect in association with any required pharmaceutical carrier. The methods of the present invention contemplate single as well as multiple administrations, given either simultaneously or over an extended period of time.

Tf-like antigens can be administered via standard routes, including the oral, ophthalmic nasal, topical, parenteral injections (e.g., intravenous, intraperitoneal, intradermal, subcutaneous or intramuscular), as well as direct injection to a preselected tissue site.

The following example further illustrates the invention.

### EXAMPLE

Sera Evaluated

The following sera were evaluated in the assay described in this example: 35 sera from normal females, 10 sera patients with clinically confirmed SLE, and 9 sera from patients having SS.

Immunometric Assay

Direct immunometric assay was performed as follows. Synthetic serine Tf, serine 2,3 sialylated Tf, serine 2,6 sialylated Tf, and serine 2,3 and 2,6 disialylated Tf antigens covalently linked separately to polystyrene microtiter plates, washed, then blocked with a non-proteinaceous buffer, poly-vinyl-pyrrolidone. Following incubation and washing, wells were incubated with HRP-conjugated class specific second antibodies. Followng washing, second antibody binding was detected in chromogenically following the addition of substrate (Ultra-TNB, Pierce Biotechnology Inc, Rockford, Ill.).

Data Analysis

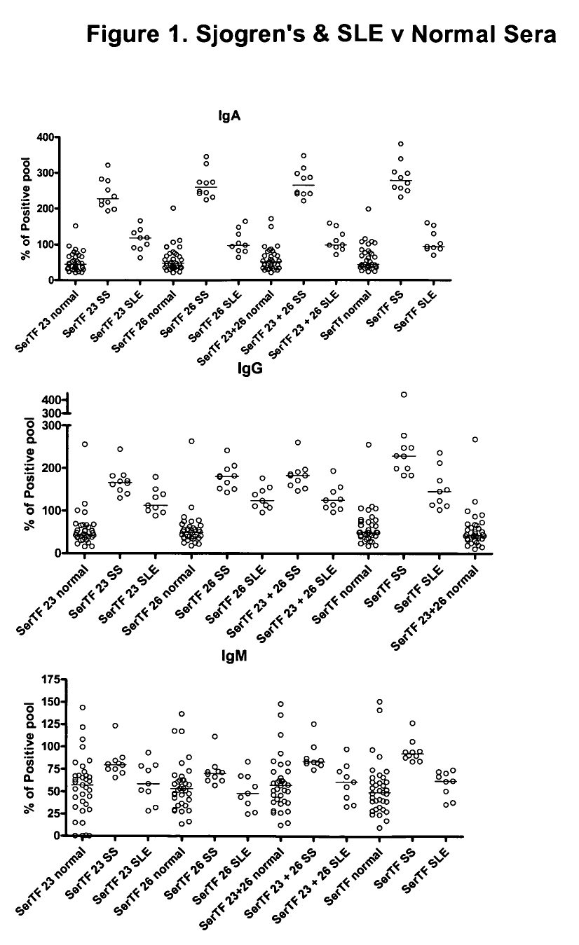

The mean amounts of secondary antibody binding for IgG, IgA and IgM of each triplicate serum sample were determined. The data are shown in FIG. 1.

As determined by Mann-Whitney non-parametric tests, the median IgG values of both patient groups are significantly elevated, whilst the IgA medians are increased in Sjögren's sera compared to the normal controls but to a much lesser extent in SLE. The median IgM value of the SS and SLE group shows only minor changes from the normal population. The elevated IgA responses in SS show complete separation from those seen in the SLE group. Thus, these two diseases can be differentially diagnosed in the same set of immunometric estimations. Distinction between SLE, SS, and normal groupings can be made by setting cut off values for IgG and/or IgA and/or IgM, based on range against 1 or more controls.

A discriminant function can correlate IgG and IgA levels in a linear manner. Examples of a discriminant function that relates IgG and IgA levels in a linear manner include, but are not limited to, the following formula:

$$Discriminant value = - 4.577 + 0.011 ( 2 , 3 sialyl Tf ) - 0.031 ( 2 , 6 sialylTf IgA ) + 0.071 ( 2 , 6 sialylTf IgM ) + 0.051 ( 2 , 3 \; 2 , 6 disialyl Tf IgG ) + 0.003 ( 2 , 3 \; 2 , 6 disialyl Tf IgM ) + 0.058 ( Tf\_IgA ) - 0.030 ( Tf IgG ) .$$

This represents a discriminant function that differentiates between normal individuals and SLE. After applying this formula to the values obtained on antibody reactivity, a positive value indicates SLE, whereas a zero or negative value indicates no disease. There are similar discriminant functions that can be used to differentiate between SLE and SS, and SS and normal individuals.

Applying this formula to the example data gives a specificity of 97.6% (i.e. 1 control is detected as a positive) and a sensitivity of 100% with complete separation of SS and SLE patients.

Alternatively, a discriminant function can relate IgG, IgA, and IgM levels in a multiplicative or geometric manner.

According to the present invention, the value of a discriminant function can be determined based on the IgG and IgA levels of a test subject. Such value is then compared to a value determined based on normal IgG and IgA levels, i.e., IgG and IgA levels of normal individuals. When comparing the value of a discriminant function derived form a test subject to a value determined from normal individuals, an increase in the value is indicative of a relevant disease in the test subject.

It should be noted that the value determined from normal individuals can be a single value, such as a median or mean value, or a range of values. Such value can be determined by employing tissue or body fluid samples from normal individuals in assays run side-by-side with the assay on a test sample. Alternatively, such value can be a predetermined value or range of values, in which case, the discriminant function value of a test subject should be determined in the same manner as the value determined from normal individuals—that is, using the same assay for determining the IgG and IgA levels, and the same formula of discriminant function.

Improvement Over Current Diagnostic Assays

The specificities and sensitivities of the discriminant function described above were compared to the currently available diagnostic assays: an ELISA assay using antibodies directed against double stranded DNA for diagnosing SLE, and an ELISA assay using antibodies directed against the antigen, SSA/Ro, for diagnosing SS.

Using the discriminant function as described above, and a cut-off value of zero, resulted in the assay characteristics presented in Table 2.

As also shown in Table 2, the sensitivities and specificities of the immunometric assay are improved as compared to an anti-dsDNA assay for diagnosing SLE and an anti-Ro/SS-A assay for diagnosing SS.

TABLE 2
Comparison of sensitivities and specificities between the Anti-Tf antibody
ELISA and the two most diagnostic single tests for SLE and SS.

| | SLE | | Sjögren's | |
|---|---|---|---|---|
| | % | | | % |
| Diagnostic Test | Sensitivity | % Specificity | % Sensitivity | Specificity |
| Anti-Tf | 100 | 97.14 | 100 | 87 |
| Anti-dsDNA | 60-70 | 95 | | |
| Anti-Ro/SS-A | | | 70 | Not Specific |

1. A method of diagnosing an autoimmune disease selected from the group consisting of lupus and Sjögren's syndrome in a subject suspected of or at risk of having an autoimmune disease selected from the group consisting of lupus and Sjögren's syndrome, comprising detecting a difference between the level of at least one antibody class or subclass in the subject that is specific for Tf-like antigens and a normal level of said antibody class or subclass, and correlating said difference with a diagnosis of an autoimmune disease selected from the group consisting of lupus and Sjögren's syndrome in the subject.

2. The method of claim 1, wherein the at least one antibody class is IgG or a subclass thereof, and wherein the diagnosis is based on detection of a higher level of anti-Tf IgG or subclass thereof in the subject as compared to a normal level of anti-Tf IgG or subclass thereof.

3. The method of claim 1, wherein the at least one antibody class is IgA or a subclass thereof, and wherein the diagnosis is based on detection of a higher level of anti-Tf IgA or subclass thereof in the subject as compared to a normal level of anti-Tf IgA or subclass thereof.

4. The method of claim 2 wherein the method further comprises detecting a difference between the level of an IgA antibody class or subclass in the subject that is specific for Tf-like antigens and a normal level of an IgA antibody class or subclass in the subject that is specific for Tf-like antigens, and correlating said difference with a diagnosis of an autoimmune disease selected from the group consisting of lupus and Sjögren's syndrome in the subject.

5. The method of claim 3 wherein the method further comprises detecting a difference between the level of an IgG antibody class or subclass in the subject that is specific for Tf-like antigens and a normal level of an IgG antibody class or subclass in the subject that is specific for Tf-like antigens, and correlating said difference with a diagnosis of an autoimmune disease selected from the group consisting of lupus and Sjögren's syndrome in the subject.

6. A method of diagnosing in a subject an autoimmune disease selected from the group consisting of lupus and Sjögren's syndrome, comprising:
a) contacting an antibody-containing tissue sample or an antibody-containing fluid sample from a subject suspected of or at risk of having an autoimmune disease selected from the group consisting of lupus and Sjögren's syndrome, with an antigen comprising a Tf-like antigen;
b) detecting autoantibody reactivity of the sample to the antigen;
c) determining a level of autoantibody reactivity of the sample relative to a normal anti-TF antibody level, and
d) correlating the level of autoantibody reactivity of the sample with a diagnosis of a presence or absence in the subject of an autoimmune disease selected from the group consisting of lupus and Sjögren's syndrome.

7. The method of claim 6, wherein the diagnosing comprises diagnosing a presence of an autoimmune disease selected from the group consisting of lupus and Sjögren's syndrome when the level of autoantibody reactivity of the sample is greater than the normal anti-TF antibody level.

8. The method of claim 6, wherein the diagnosing comprises diagnosing an absence of an autoimmune disease selected from the group consisting of lupus and Sjögren's Syndrome when the level of autoantibody reactivity of the sample is similar to normal anti-TF antibody level.

9. The method of claim 6 wherein the detecting comprises detecting of one or both of anti-Tf-IgG and anti-Tf IgA autoantibody reactivity of the sample to a Tf-like antigen;
wherein the determining comprises determining a level of one or both of anti-Tf-IgG and anti-Tf IgA autoantibody reactivity of the sample relative to normal anti-TF antibody level; and
wherein the correlating comprises correlating the level of one or both of anti-Tf-IgG and anti-Tf IgA autoantibody reactivity of the sample with a diagnosis of a presence or absence in the subject of an autoimmune disease selected from the group consisting of lupus and Sjögren's syndrome.

10. The method of claim 9 wherein the diagnosing comprises diagnosing a presence of an autoimmune disease selected from the group consisting of lupus and Sjögren's Syndrome when the level of one or both of anti-Tf-IgG and anti-Tf IgA autoantibody reactivity of the sample is greater than a normal level of anti-Tf-IgG and/or anti-Tf IgA levels.

11. The method of claim 9 wherein the diagnosing comprises diagnosing an absence of an autoimmune disease selected from the group consisting of lupus and Sjögren's Syndrome when the level of one or both of anti-Tf-IgG and anti-Tf IgA autoantibody reactivity of the sample is similar to a normal level of anti-Tf-IgG and/or anti-Tf IgA levels.

12. A method of diagnosing an autoimmune disease selected from the group consisting of lupus and Sjögren's syndrome in a subject suspected of or at risk of having an autoimmune disease selected from the group consisting of lupus and Sjögren's syndrome, comprising determining a test value of a discriminant function based on levels of at least two antibody classes or subclasses in the subject that are specific for Tf-like antigens, comparing the test value to a normal value determined from normal individuals, and detecting an increase in the test value as indicative of an autoimmune disease selected from the group consisting of lupus and Sjögren's syndrome.

13. The method of claim 12, wherein said discriminant function is a function of the level of anti-Tf IgG or subclass thereof and the level of anti-Tf IgA or subclass thereof in the subject.

14. The method of claim 12, wherein said discriminant function is a function that positively weighs levels of anti-Tf IgG or subclass thereof and levels of anti-Tf IgA or subclass thereof.

15. The method of claim 12, wherein said discriminant function relates the IgG and IgA levels, or subclasses thereof, in a linear, multiplicative or geometric manner.

16. The method of claim 12, wherein said discriminant function comprises the formula: Discriminant value=−4.577+0.011(2,3 sialyl Tf)−0.031(2,6 sialylTf IgA)+0.071(2,6 sialylTf IgM)+0.051(2,3 2,6 disialyl Tf IgG)+0.003(2,3 2,6 disialyl Tf IgM)+0.058(Tf_IgA)−0.030(TfIgG).

17. The method of claim 1 wherein the autoimmune disease is lupus.

18. The method of claim 17 wherein the lupus comprises systemic lupus erythematosus.

19. The method of claim 17 wherein the lupus comprises discoid lupus.

20. The method of claim 17 wherein the lupus comprises lupoid hepatitis.

21. The method of claim 1 wherein the autoimmune disease is Sjögren's syndrome.

22. The method of claim 21 wherein the Sjögren's syndrome comprises primary Sjögren's syndrome.

23. The method of claim 21 wherein the Sjögren's syndrome comprises secondary Sjögren's syndrome.

24. The method of claim 1 wherein the Tf-like antigen comprises one or more Galβ1-3GalNAc disaccharide moiety.

25. The method of claim 1, wherein the level of at least one antibody class or subclass in the subject is determined in an antibody-containing tissue or antibody-containing body fluid from the subject, wherein the antibody-containing tissue or antibody-containing body fluid is selected from the group consisting of blood, serum, plasma, saliva, tears, sweat, peritoneal fluid, or vaginal secretion.

26. The method of claim 6, wherein the antibody-containing tissue or antibody-containing body fluid is selected from the group consisting of blood, serum, plasma, saliva, tears, sweat, peritoneal fluid, or vaginal secretion.

27. The method of claim 1 wherein the level of autoantibody reactivity of the sample relative to a control is determined in an immunoassay.

28. The method of claim 27, wherein said immunoassay employs an antibody-containing sample obtained from the subject to be diagnosed.

29. The method of claim 28, wherein said sample is a tissue or body fluid sample.

30. The method of claim 29, wherein said body fluid is selected from blood, serum, plasma, saliva, tears, sweat, peritoneal fluid, or vaginal secretion.

31. A method of treating an autoimmune disease selected from the group consisting of lupus and Sjögren's syndrome, comprising administering to a subject with one or more of lupus and Sjögren's syndrome an amount effective of an antibody that specifically binds a Tf-like antigen to treat the autoimmune disease.

32. The method of claim 31, wherein the antibody comprises a monoclonal antibody.

33. The method of claim 31, wherein the antibody comprises a humanized antibody.

34. The method of claim 31, wherein said Tf-like antigen comprise the Galβ1-3GalNAc disaccharide moiety.

35. The method of claim 31 wherein the antibody is administered to the patient via an oral, parenteral, or subcutaneous route.

36. A method of treating an autoimmune disease selected from the group consisting of lupus and Sjögren's Syndrome, comprising administering to a subject with one or more of lupus and Sjögren's Syndrome an amount effective of a Tf-like antigen to treat the autoimmune disease.

37. The method of claim 36, wherein the Tf-like antigen comprises one or more Galβ1-3GalNAc disaccharide moiety.

38. The method of claim 36, wherein the Tf-like antigen is administered to the subject via an oral, parenteral, or subcutaneous route.
